SEQUENCE LISTING (2 sequences)

SEQ ID NO: 1 - DNA, 4315 bp, Homo sapiens
ggaaattgttcctcgtctgataagacaacagtggagaaaggacgcatgctgtttcttagggacacggctggcttccagatatgaccatgtatttgtggcttaaactcttggcatttggctttgcctttctggacacagaagtatttgtgacagggcaaagcccaacaccttcccccactggattgactacagcaaagatgcccagtgttccactttcaagtgaccccttacctactcacaccactgcattctcacccgcaagcacctttgaaagagaaaatgacttctcagagaccacaacttctcttagtccagacaatacttccacccaagtatccccggactctttggataatgctagtgcttttaataccacaggtgtttcatcagtacagacgcctcaccttcccacgcacgcagactcgcagacgccctctgctggaactgacacgcagacattcagcggctccgccgccaatgcaaaactcaaccctaccccaggcagcaatgctatctcagatgtcccaggagagaggagtacagccagcacctttcctacagacccagtttccccattgacaaccaccctcagccttgcacaccacagctctgctgccttacctgcacgcacctccaacaccaccatcacagcgaacacctcagatgcctaccttaatgcctctgaaacaaccactctgagcccttctggaagcgctgtcatttcaaccacaacaatagctactactccatctaagccaacatgtgatgaaaaatatgcaaacatcactgtggattacttatataacaaggaaactaaattatttacagcaaagctaaatgttaatgagaatgtggaatgtggaaacaatacttgcacaaacaatgaggtgcataaccttacagaatgtaaaaatgcgtctgtttccatatctcataattcatgtactgctcctgataagacattaatattagatgtgccaccaggggttgaaaagtttcagttacatgattgtacacaagttgaaaaagcagatactactatttgtttaaaatggaaaaatattgaaacctttacttgtgatacacagaatattacctacagatttcagtgtggtaatatgatatttgataataaagaaattaaattagaaaaccttgaacccgaacatgagtataagtgtgactcagaaatactctataataaccacaagtttactaacgcaagtaaaattattaaaacagattttgggagtccaggagagcctcagattattttttgtagaagtgaagctgcacatcaaggagtaattacctggaatccccctcaaagatcatttcataattttaccctctgttatataaaagagacagaaaaagattgcctcaatctggataaaaacctgatcaaatatgatttgcaaaatttaaaaccttatacgaaatatgttttatcattacatgcctacatcattgcaaaagtgcaacgtaatggaagtgctgcaatgtgtcatttcacaactaaaagtgctcctccaagccaggtctggaacatgactgtctccatgacatcagataatagtatgcatgtcaagtgtaggcctcccagggaccgtaatggcccccatgaacgttaccatttggaagttgaagctggaaatactctggttagaaatgagtcgcataagaattgcgatttccgtgtaaaagatcttcaatattcaacagactacacttttaaggcctattttcacaatggagactatcctggagaaccctttattttacatcattcaacatcttataattctaaggcactgatagcatttctggcatttctgattattgtgacatcaatagccctgcttgttgttctctacaaaatctatgatctacataagaaaagatcctgcaatttagatgaacagcaggagcttgttgaaagggatgatgaaaaacaactgatgaatgtggagccaatccatgcagatattttgttggaaacttataagaggaagattgctgatgaaggaagaccttttctggctgaatttcagagcatcccgcgggtgttcagcaagtttcctataaaggaagctcgaaagccctttaaccagaataaaaaccgttatgttgacattcttccttatgattataaccgtgttgaactctctgagataaacggagatgcagggtcaaactacataaatgccagctatattgatggtttcaaagaacccaggaaatacattgctgcacaaggtcccagggatgaaactgttgatgatttctggaggatgatttgggaacagaaagccacagttattgtcatggtcactcgatgtgaagaaggaaacaggaacaagtgtgcagaatactggccgtcaatggaagagggcactcgggcttttggagatgttgttgtaaagatcaaccagcacaaaagatgtccagattacatcattcagaaattgaacattgtaaataaaaaagaaaaagcaactggaagagaggtgactcacattcagttcaccagctggccagaccacggggtgcctgaggatcctcacttgctcctcaaactgagaaggagagtgaatgccttcagcaatttcttcagtggtcccattgtggtgcactgcagtgctggtgttgggcgcacaggaacctatatcggaattgatgccatgctagaaggcttggaagccgagaacaaagtggatgtttatggttatgttgtcaagctaaggcgacagagatgcctgatggttcaagtagaggcccagtacatcttgatccatcaggctttggtggaatacaatcagtttggagaaacagaagtgaatttgtctgaattacatccatatctacataacatgaagaaaagggatccacccagtgagccgtctccactagaggctgaattccagagacttccttcatataggagctggaggacacagcacattggaaatcaagaagaaaataaaagtaaaaacaggaattctaatgtcatcccatatgactataacagagtgccacttaaacatgagctggaaatgagtaaagagagtgagcatgattcagatgaatcctctgatgatgacagtgattcagaggaaccaagcaaatacatcaatgcatcttttataatgagctactggaaacctgaagtgatgattgctgctcagggaccactgaaggagaccattggtgacttttggcagatgatcttccaaagaaaagtcaaagttattgttatgctgacagaactgaaacatggagaccaggaaatctgtgctcagtactggggagaaggaaagcaaacatatggagatattgaagttgacctgaaagacacagacaaatcttcaacttatacccttcgtgtctttgaactgagacattccaagaggaaagactctcgaactgtgtaccagtaccaatatacaaactggagtgtggagcagcttcctgcagaacccaaggaattaatctctatgattcaggtcgtcaaacaaaaacttccccagaagaattcctctgaagggaacaagcatcacaagagtacacctctactcattcactgcagggatggatctcagcaaacgggaatattttgtgctttgttaaatctcttagaaagtgcggaaacagaagaggtagtggatatttttcaagtggtaaaagctctacgcaaagctaggctaggcatggtttccacattcgagcaatatcaattcctatatgacgtcattgccagcacctaccctgctcagaatggacaagtaaagaaaaacaaccatcaagaagataaaattgaatttgataatgaagtggacaaagtaaagcaggatgctaattgtgttaatccacttggtgccccagaaaagctccctgaagcaaaggaacaggctgaaggttctgaacccacgagtggcactgaggggccagaacattctgtcaatggtcctgcaagtccagctttaaatcaaggttcataggaaaagacataaatgaggaaactccaaacctcctgttagctgttatttctatttttgtagaagtaggaagtgaaaataggtatacagtggattaattaaatgcagcgaaccaatatttgtagaagggttatattttactactgtggaaaaatatttaagatagttttgccagaacagtttgtacagacgtatgcttattttaaaattttatctcttattcagtaaaaaacaacttctttgtaatcgttatgagtgtatatgtatgtgtgtatgggtgtgtgtttgtgtgagagacagagaaagagagagaattc

SEQ ID NO: 2 - protein, 1306 aa, Homo sapiens
MTMYLWLKLLAFGFAFLDTEVFVTGQSPTPSPTGLTTAKMPSVPLSSDPLPTHTTAFSPASTFERENDFSETTTSLSPDNTSTQVSPDSLDNASAFNTTGVSSVQTPHLPTHADSQTPSAGTDTQTFSGSAANAKLNPTPGSNAISDVPGERSTASTFPTDPVSPLTTTLSLAHHSSAALPARTSNTTITANTSDAYLNASETTTLSPSGSAVISTTTIATTPSKPTCDEKYANITVDYLYNKETKLFTAKLNVNENVECGNNTCTNNEVHNLTECKNASVSISHNSCTAPDKTLILDVPPGVEKFQLHDCTQVEKADTTICLKWKNIETFTCDTQNITYRFQCGNMIFDNKEIKLENLEPEHEYKCDSEILYNNHKFTNASKIIKTDFGSPGEPQIIFCRSEAAHQGVITWNPPQRSFHNFTLCYIKETEKDCLNLDKNLIKYDLQNLKPYTKYVLSLHAYIIAKVQRNGSAAMCHFTTKSAPPSQVWNMTVSMTSDNSMHVKCRPPRDRNGPHERYHLEVEAGNTLVRNESHKNCDFRVKDLQYSTDYTFKAYFHNGDYPGEPFILHHSTSYNSKALIAFLAFLIIVTSIALLVVLYKIYDLHKKRSCNLDEQQELVERDDEKQLMNVEPIHADILLETYKRKIADEGRLFLAEFQSIPRVFSKFPIKEARKPFNQNKNRYVDILPYDYNRVELSEINGDAGSNYINASYIDGFKEPRKYIAAQGPRDETVDDFWRMIWEQKATVIVMVTRCEEGNRNKCAEYWPSMEEGTRAFGDVVVKINQHKRCPDYIIQKLNIVNKKEKATGREVTHIQFTSWPDHGVPEDPHLLLKLRRRVNAFSNFFSGPIVVHCSAGVGRTGTYIGIDAMLEGLEAENKVDVYGYVVKLRRQRCLMVQVEAQYILIHQALVEYNQFGETEVNLSELHPYLHNMKKRDPPSEPSPLEAEFQRLPSYRSWRTQHIGNQEENKSKNRNSNVIPYDYNRVPLKHELEMSKESEHDSDESSDDDSDSEEPSKYINASFIMSYWKPEVMIAAQGPLKETIGDFWQMIFQRKVKVIVMLTELKHGDQEICAQYWGEGKQTYGDIEVDLKDTDKSSTYTLRVFELRHSKRKDSRTVYQYQYTNWSVEQLPAEPKELISMIQVVKQKLPQKNSSEGNKHHKSTPLLIHCRDGSQQTGIFCALLNLLESAETEEVVDIFQVVKALRKARPGMVSTFEQYQFLYDVIASTYPAQNGQVKKNNHQEDKIEFDNEVDKVKQDANCVNPLGAPEKLPEAKEQAEGSEPTSGTEGPEHSVNGPASPALNQGS

(19) United States
(12) Patent Application Publication
(10) Pub. No.: US 2021/0269526 A1
(43) Pub. Date: Sep. 2, 2021

(54) BISPECIFIC AFFINITY REAGENT AND RELATED METHODS FOR PRETARGETED RADIOIMMUNOTHERAPY AGAINST CD45+ CELL

(71) Applicant: FRED HUTCHINSON CANCER RESEARCH CENTER, Seattle, WA (US)

(72) Inventor: Johnnie J. OROZCO, Seattle, WA (US)

(73) Assignee: FRED HUTCHINSON CANCER RESEARCH CENTER, Seattle, WA (US)

(21) Appl. No.: 17/260,205

(22) PCT Filed: Jul. 16, 2019
(86) PCT No.: PCT/US2019/042065

§ 371 Date: Jan. 13, 2021

Related U.S. Application Data
(60) Provisional application No. 62/698,643, filed on Jul. 16, 2018.

(51) Int. Cl. C07K 16/28; C07K 16/44; A61K 51/04; A61P 35/02

(52) U.S. Cl. CPC C07K 16/289; C07K 16/44; A61K 51/0482; A61P 35/02; A61K 2039/505; C07K 2317/622; C07K 2317/626; C07K 2317/55; C07K 2317/31

(57) ABSTRACT

The disclosure is directed to bispecific affinity reagents, e.g., fusion proteins, that specifically bind to CD45 and radioactive ligands. The disclosure also addresses methods of use of the bispecific affinity reagents to specifically target cells with aberrant CD45 expression for therapy.

### CROSS-REFERENCE TO RELATED APPLICATION

This application claims the benefit of U.S. Provisional Application No. 62/698,643, filed Jul. 16, 2018, the entire disclosure of which is incorporated by reference herein in its entirety.

### STATEMENT OF GOVERNMENT LICENSE RIGHTS

This invention was made with Government support under R01CA109663 and R01CA136639 awarded by the National Institutes of Health. The Government has certain rights in the invention.

### BACKGROUND

Acute myeloid leukemia (AML) is a very aggressive cancer of the blood, associated with poor outcomes, and only a 25% five year overall survival. Incidence of AML increases with age, with median age of diagnosis 67 years, which makes treatment challenging as elderly patients may have other medical co-morbidities that make them ineligible for intensive treatment. Unfortunately, intensive chemotherapy options have been historically reserved for younger, fit patients, as elderly patients may be too frail to tolerate toxicity associated with intensive chemotherapy options. For decades now the standard of care intensive chemotherapy option has been infusional cytarabine (usually 100 to 200 mg/m2 per day) for 7 days, combined with an anthracycline, like daunorubicin for 3 days, nicknamed “7+3 therapy”. The standard treatment option of 7+3 is associated with pancytopenia, which places the patient at risk of potentially fatal infections and/or bleeding events. One study reported on over 1500 AML patients who received 7+3 per different Southwest Oncology Group (SWOG) protocols, where response assessment (when counts recover usually 3-5 weeks after induction chemotherapy) showed a CR rate of 48%, with an early death rate of 9%. Other regimens have attempted to improve upon the 7+3 currently accepted standard of care, without significant results, with higher doses of cytarabine, combination chemotherapy like ICE, FLAG-Ida.

Therapeutic efficacy could be improved, with less toxicity, if anti-neoplastic radiation could be selectively focused on malignant cells. Thus, antibodies specific for tumor-associated antigens, conjugated to toxins, drugs or radioactive isotopes provide an appealing targeted treatment platform. Radiolabeled antibodies are particularly suitable for hematologic malignancies because 1) leukemias and lymphomas are especially sensitive to radiation therapy; and 2) surface antigens and potential targets are well-characterized, many with multiple effective antibodies. More importantly, clinically meaningful results have been achieved in clinical trials using radiolabeled antibodies through radioimmunotherapy (RIT). Although there are multiple potential targets for AML, CD45 has recently been targeted in RIT studies because of its expression at a high copy number on cell surfaces of nearly all hematopoietic cells, but negligibly expression on non-hematopoietic tissues. CD45 RIT has been reported to effectively treat both lymphomas and leukemias in pre-clinical studies, and HCT clinical trials for high risk disease, with a 63% 3-year survival in very high risk patients who were ineligible for other HCT protocols.

Despite the improved outcomes in patients with high-risk disease, RIT remains a challenge to implement for a variety of logistical and theoretical reasons. For example, there remains concern that non-specific irradiation of uninvolved organs occurs as the radioimmunoconjugate circulates before binding to its target, leading to excessive toxicity.

Accordingly, despite the advances in the specific targeting of hematologic malignancies, there remains a need for compositions and methods that safely and specifically target cancerous cells that safely minimize off-target toxicity. The present disclosure addresses these and related needs.

### DESCRIPTION OF THE DRAWINGS

The foregoing aspects and many of the attendant advantages of this invention will become more readily appreciated as the same become better understood by reference to the following detailed description, when taken in conjunction with the accompanying drawings, wherein:

FIGS. 1A-1D illustrate the structure and characterization of representative embodiment of an anti-murine CD45 bispecific antibody affinity reagent (i.e., fusion protein) (referred to as 30F11-Fc-C825) provided by the present disclosure. FIG. 1A is a cartoon depiction of a representative embodiment of the bispecific affinity reagent (i.e., fusion protein) of the present disclosure. The illustrated configuration includes an antibody with two antigen binding fragment (Fab) domains having binding affinity for CD45 (blue/green), each anti-CD45 Fab being fused to an scFv with binding affinity for a radiolabeled chelator such as yttrium (Y)-DOTA (yellow). The heavy chain variable region (VH) from the anti-mCD45 antibody (30F11) is in between the signal peptide (SP) and hIgG1 Fc hinge sequences. The light chain variable region (VL) of the anti-mCD45 (30F11), in addition to the anti-⁹⁰Y-DOTA capturing (C825) disulfide (ds) stabilized scFv. FIG. 1B is a cartoon depiction illustrating the schematic design of gene constructs encoding the antibody bispecific affinity reagent (i.e., fusion protein) illustrated in FIG. 1A. An anti-murine CD45 (30F11) V_{L} gene and a yttrium (Y)-DOTA capturing (C825) disulfide-stabilized scFv (ds-scFv) gene were fused to a human kappa fragment at amino or carboxyl end, respectively (pFUSE2ss-CLIg-hk, InvivoGen). A V_{H} from the anti-mCD45 antibody (30F11) was fused to a human IgG1 fragment as indicated (pFUSEss-CHIg-hG1, InvivoGen). The bispecific Fc fusion polypeptides were expressed in HEK cells, spontaneously pairing between light and heavy chains and forming an IgG1-like fusion antibody, and secreted in the growth media. FIG. 1C is a Western blot of 30F11 Heavy and Light chains confirming production of the antibody bispecific affinity reagent (i.e., fusion protein) illustrated in FIGS. 1A and 1B. Heavy and light chains were produced as described in the Examples, and the purified bispecific 30F11-Fc-C825 antibody was analyzed by SDS-PAGE. Lane 1 contains the ladder in (kDa), with the following lane showing the boiled and denatured heavy and light chains of the bispecific antibody with beta-mercaptoethanol, with the last lane showing the bispecific antibody in non-denaturing (non-reduced) conditions. FIG. 1D graphically illustrates the confirmation of binding affinity of the antibody bispecific affinity reagent (i.e., fusion protein) illustrated in FIGS. 1A and 1B. Functional binding to mCD45+ target cells (EL4) and Y-DOTA was assayed by flow cytometry. EL4 cells were incubated with Y-DOTA-biotin alone, or with blocking monoclonal Ab 30F11 (30F11 mAb) and Y-DOTA-biotin with either bispecific Ab from batch 1 (30F11-Fc-C825) or batch 2 (30F11-Ab-C825). Other EL4 cells were also incubated with Y-DOTA-biotin and bispecific Ab from batch 1 (30F11-Fc-C825) or batch 2 (30F11-Ab-C825) or non-binding negative bispecific Ab (CC49-Fc-C825). Cells were washed after incubated and assayed with PE-labeled anti-streptavidin secondary Ab (PE-SA) that bound to biotin from Y-DOTA-biotin.

FIGS. 2A and 2B illustrate the biodistribution and efficacy of anti-murine CD45 antibody bispecific affinity reagent (i.e., fusion protein) (30F11-Fc-C825) in syngeneic leukemia bearing mice. FIG. 2A graphically illustrates biodistribution of the CD45+ cells in the murine model as determined by use of the antibody bispecific affinity reagent (i.e., fusion protein) illustrated in FIGS. 1A and 1B. Five mice per group (B6SJLF1/J) with disseminated murine myeloid leukemia (105 SJL cells) for 2 days were given 1.4 nmol of either 30F11-Fc-C825 or a non-binding (CC49-Fc-C825) negative control bispecific antibody, followed by DOTAY-dextran clearing agent to remove any unbound, circulating bispecific antibody 22 hours after the bispecific. Two hours after clearing agent, mice were given 50 μCi of 90Y-DOTA-biotin. Mice were sacrificed at 6 or 24 hours after radiolabeled DOTA-biotin injections, organs harvested and tissues assayed for % of injected dose per gram of organ (% ID/g). FIG. 2B graphically illustrates survival of mice bearing disseminated SJL leukemia (10 per group), treated with 1.4 nmol of 30F11-Fc-C825 or CC49-Fc-C825 FP, followed by clearing agent (CA), before 800, 1000, or 1200 μCi of ⁹⁰Y-DOTA-biotin. Ten mice per group (B6SJLF1/J) were given SJL leukemia, bispecific antibody (30F11-Fc-C825, non-targeting CC49-Fc-C825, or none (no treatment control)), and clearing agent per the sequence described above in biodistribution studies. Two hours later groups of mice were given escalating doses of (800-1500 of 90Y-DOTA-biotin and followed for efficacy.

FIGS. 3A-3E illustrate the structure and characterization of anti-human CD45 bispecific Fc antibody affinity reagent (i.e., fusion protein) (BC8-Fc-C825). FIG. 3A is a cartoon depiction of another representative embodiment of the bispecific affinity reagent (i.e., fusion protein) of the present disclosure that is an anti-human CD45 BC8 bispecific (anti-hCD45×anti-Y-DOTA) antibody construct. The illustrated configuration includes antibody heavy constant chain (C_{H}) domains (disposed centrally in the construct) fused to scFv having binding affinity for CD45 or for a radiolabeled chelator such as yttrium (Y)-DOTA. FIG. 3B is a cartoon depiction illustrating the schematic of a gene construct encoding the bispecific Fc antibody affinity reagent (i.e., fusion protein) illustrated in FIG. 3A. The gene coding for the anti-human CD45 scFv and the gene coding for the Y-DOTA capturing C825 disulfide-stabilized scFv (ds-scFv) were added onto the human IgG1 Fc fragment at the amino and carboxyl ends, respectively. Key restriction digest sites for cloning/linearization are shown relative to other features. An N-linked glycosylation containing linker (NLG) was also incorporated between two functional domains Fc and C825 ds-scFv. The diagram is not drawn to scale. FIG. 3C is a Western blot of the bispecific Fc fusion polypeptides illustrated in FIGS. 3A and 3B. The construct illustrated in FIG. 3B was expressed in CHO-DG44 cells, and the bispecific BC8-Fc-C825 fusion antibody protein was purified from supernatant, as spontaneously formed dimers as seen by SDS-PAGE analysis. Lane 1 shows SeeBlue Plus2 protein ladder markers in kilodaltons; lane 2 shows a non-reduced, boiled bispecific; and lane 3 shows the monomer of the bispecific protein boiled and reduced with 2-mercptoethanol. FIG. 3D graphically illustrates the confirmation of binding affinity of the bispecific Fc affinity reagent (i.e., fusion protein) illustrated in FIGS. 3A and 3B for CD45-expressing cells. Functional binding to hCD45+ target cells (Ramos cells) and Y-DOTA was assayed by flow cytometry. Ramos cells were incubated with no Ab, BC8 mAb alone, BC8-Fc-C825 bispecific Ab alone or with competing BC8 mAb 10:1. Cells then were washed and assayed with PE-labeled anti-streptavidin secondary Ab (PE-SA) that bound to biotin from Y-DOTA-biotin. Functional binding to Y-DOTA was confirmed by sandwich ELISA assay. FIG. 3E graphically illustrates the confirmation of binding affinity of the bispecific Fc affinity reagent (i.e., fusion protein) illustrated in FIGS. 3A and 3B for Y-DOTA. A 96-well plate was first coated with Y-DOTA in BSA blocking buffer, and after washing with 2% BSA in PBS, wells were treated with bispecific BC8-Fc-C825 (or non-binding negative control bispecific LDL-Fc-C825) starting at 16 μg/ml followed by serial dilution. After washing, wells were treated with horseradish peroxidase (HRP)-anti-human Fc secondary Ab followed by colorimetric reagent 3,3′,5,5′-tetramethylbenzidine (TMB) as measured by A450.

FIGS. 4A-4E illustrate the biodistribution and blood clearance of 90Y-DOTA-biotin using BC8-Fc-C825. FIG. 4A graphically illustrates the clearance of the BC8 bispecific antibody affinity reagent (i.e., fusion protein) clearance. Athymic nude mice bearing right flank HEL subcutaneous tumors (5 mice/group) were given 1.4 nmol (or 2.8 nmol where stated) of BC8-Fc-C825, streptavidin-modified 1st step (BC8-SAV), or negative control non-binding CC49-Fc-C825 bispecific antibody, followed by 0 (panel A) or 5 μg of DOTAY-dextran clearing agent (CA) 23 hours later, and finally 1 hour later ⁹⁰Y-DOTA-biotin. Mice had peripheral blood drawn (20 μL) by retro-orbital venous sampling at different time points (0 to 20 hours), and assayed by gamma counter to assess percent of injected dose per gram of blood (% ID/g) to characterize blood clearance. FIGS. 4B-4E graphically illustrate the results of biodistribution studies where mice were treated as above, and euthanized at 4, 24, 48 or 96 hours after ⁹⁰Y-DOTA-biotin injection, organs and subcutaneous tumors harvested and assayed by gamma counter to calculate the % ID/g of organ tissue (sm int: small intestine; lg int: large intestine).

FIGS. 5A-5D illustrate the therapeutic efficacy of BC8-Fc-C825 compared to BC8-SAV biotin-streptavidin approach. FIGS. 5A and 5B illustrate results of assays where athymic nude mice (10 mice/group) bearing flank subcutaneous HEL xenograft tumors (˜100 mm3) were given 1.4 nmol of BC8-SAV, BC8-Fc-C825, BHV1-SAV or CC49-Fc-C825 (non-targeting controls) at day 8 post HEL-implantation. Approximately 22 hours later, mice were injected with 5 μg of DOTAY-dextran clearing agent, followed 2 hours later by 1400 μCi ⁹⁰Y-DOTA-biotin. Mice were followed for tumor volumes (FIG. 5A) where curves were truncated at first death in that group for excessive tumor size, and overall survival (FIG. 5B). There were three and four deaths in the BC8-SAV and BC8.C825 groups, respectively, due to early toxicity. Mantel-Cox log-rank testing on the survival outcomes shows that the BC8-C825 group is statistically different from the CC49-C825 group, with a p value of 0.0244. The BC8-C825 and BC8-SAV survival data are not statistically significant, with a p value of 0.3768. FIGS. 5C and 5D illustrate the results of an additional set of assays that were performed to characterize a dose response to BC8-Fc-C825. Athymic nude mice (10 mice/group) were given flank subcutaneous HEL xenograft tumors. When tumors were approximately 100 mm³ (2 days post HEL-implantation) mice were given 1.4 nmol of BC8-Fc-C825 or non-targeting CC49-Fc-C825 bispecific antibody. DOTAY-dextran clearing agent (5 μg) was injected 23 hours later, followed 1 hour later by 1000, 1200 or 1400 μCi ⁹⁰Y-DOTA-biotin. Tumor volume curves were truncated at first death in group due to excessive tumor size (FIG. 5C) and overall survival by radioactivity amount (FIG. 5D). There were three deaths in each treatment group due to toxicity.

### DETAILED DESCRIPTION

To address concerns of toxicity related to radioimmunotherapy (RIT), two-step pre-targeted RIT (PRIT) approaches have been developed to separate the delivery of radioactivity from the initial targeting step. Briefly, in the first step a non-radiolabeled modified tumor-specific antibody is infused and allowed to localize to target sites. In the second step, the radiolabeled moieties, which has a high affinity for the modified tumor-specific antibody, is administered. The radiolabeled moieties are efficiently captured and retained by the pre-targeted antibody, whereas unbound radiolabeled reagent is rapidly excreted, thereby minimizing non-specific radiation during circulation. Although many of these PRIT approaches have improved biodistributions compared to directly labeled radioimmunoconjugates, some have potentially greater immunogenicity than others. Additionally, in some PRIT approaches, such as a streptavidin-biotin approach, non-target toxicity can remain an issue. Furthermore, there have been no studies comparing their relative merits.

As described in the present disclosure, the inventors have developed a bispecific affinity reagent that permits a pre-targeted MT approach that overcomes some of the concerns associated with the streptavidin-biotin approach, including concerns of off-target toxicity. As described in more detail below, the inventors demonstrate that the bispecific constructs targeting CD45 and a radioactive ligand, yttrium (Y)-DOTA, effectively targeted radiation to target tissues in murine leukemia models, and resulted in improved overall survival. When compared to the streptavidin PRIT approach, the disclosed bispecific affinity reagent (i.e., fusion protein) construct targeting CD45 was as effective and further avoids certain issues with the extant technologies.

In accordance with the foregoing, the disclosure provides a bispecific affinity reagent comprising a first binding domain and a second binding domain. The first binding domain specifically binds to a CD45 cancer antigen, and the second binding domain specifically binds to a radioactive ligand.

As used herein, the term “bispecific” indicates multiple, distinct binding domains within the affinity reagent so as to confer specific binding of the affinity reagent to at least two different antigens (e.g., CD45 and a radioactive ligand). The term encompasses additional domains (e.g., a third binding domain) that confer specific binding to additional (e.g., a third) antigen.

Binding Domains

As used herein the term “binding domain” refers to a molecular domain, such as in a peptide, oligopeptide, polypeptide, or protein, which possesses the ability to specifically and non-covalently associate, unite, or combine with a target molecule (e.g., CD45 or radioactive ligand). A binding domain can be any naturally occurring, synthetic, semi-synthetic, or recombinantly produced binding partner for the target of interest. In some embodiments, a binding domain is or comprises functional (i.e., antigen binding) elements of an immunoglobulin or immunoglobulin-like molecule, such as an antibody or T cell receptor (TCR), aptamer, peptidobodies, antigen-binding scaffolds (e.g., DARPins, HEAT repeat proteins, ARM repeat proteins, tetratricopeptide repeat proteins, and other scaffolds based on naturally occurring repeat proteins, etc. (see, e.g., Boersma and Pluckthun, Curr. Opin. Biotechnol. 22:849-857, 2011, and references cited therein, incorporated herein by reference)), which include a functional binding domain or antigen-binding fragment thereof.

In some embodiments, one or both of the first binding domain and a second binding domain of the affinity reagent is or comprises an antibody or a functional antibody fragment. As used herein, the terms “antibody” and “antibody fragments” encompasses antibodies and fragments thereof derived from any antibody-producing mammal (e.g., mouse, rat, rabbit, and primate including human), and which specifically bind to an antigen of interest. Exemplary antibodies include polyclonal, monoclonal and recombinant antibodies; multispecific antibodies (e.g., bispecific antibodies); humanized antibodies; murine antibodies; chimeric, mouse-human, mouse-primate, primate-human monoclonal antibodies; and anti-idiotype antibodies. The antigen-binding molecule can be any intact antibody molecule or fragment thereof (e.g., with a functional antigen-binding domain).

An antibody fragment is a portion derived from or related to a full-length antibody, preferably including the complementarity-determining regions (CDRs), antigen binding regions, or variable regions thereof. Illustrative examples of antibody fragments useful in the present disclosure include Fab, Fab′, F(ab)₂, F(ab′)₂ and Fv fragments, scFv fragments, diabodies, linear antibodies, single-chain antibody molecules, multispecific antibodies formed from antibody fragments, and the like. A “single-chain Fv” or “scFv” antibody fragment comprises the V_{H} and V_{L} domains of an antibody, wherein these domains are present in a single polypeptide chain. The Fv polypeptide can further comprise a polypeptide linker between the V_{H} and V_{L} domains, which enables the scFv to form the desired structure for antigen binding. Antibody fragments can be produced recombinantly, or through enzymatic digestion.

Antibodies can be further modified to suit various uses. For example, a “chimeric antibody” is a recombinant protein that contains domains from different sources. For example, the variable domains and complementarity-determining regions (CDRs) can be derived from a non-human species (e.g., rodent) antibody, while the remainder of the antibody molecule is derived from a human antibody. A “humanized antibody” is a chimeric antibody that comprises a minimal sequence that conforms to specific complementarity-determining regions derived from non-human immunoglobulin that is transplanted into a human antibody framework. Humanized antibodies are typically recombinant proteins in which only the antibody complementarity-determining regions (CDRs) are of non-human origin.

Antibody fragments and derivatives that recognize specific epitopes can be generated by any technique known to those of skill in the art. For example, Fab and F(ab′)₂ fragments of the invention can be produced by proteolytic cleavage of immunoglobulin molecules, using enzymes such as papain (to produce Fab fragments) or pepsin (to produce F(ab′)₂ fragments). F(ab′)₂ fragments contain the variable region, the light chain constant region and the CHI domain of the heavy chain. Further, the antibodies of the present invention can also be generated using various phage display methods known in the art. Finally, the antibodies, or antibody fragments or derivatives can be produced recombinantly according to known techniques.

As used herein, the term “aptamer” refers to oligonucleic or peptide molecules that can bind to specific antigens of interest. Nucleic acid aptamers usually are short strands of oligonucleotides that exhibit specific binding properties. They are typically produced through several rounds of in vitro selection or systematic evolution by exponential enrichment protocols to select for the best binding properties, including avidity and selectivity. One type of useful nucleic acid aptamers are thioaptamers, in which some or all of the non-bridging oxygen atoms of phophodiester bonds have been replaced with sulfur atoms, which increases binding energies with proteins and slows degradation caused by nuclease enzymes. In some embodiments, nucleic acid aptamers contain modified bases that possess altered side-chains that can facilitate the aptamer/target binding.

Peptide aptamers are protein molecules that often contain a peptide loop attached at both ends to a protamersein scaffold. The loop typically has between 10 and 20 amino acids long, and the scaffold is typically any protein that is soluble and compact. One example of the protein scaffold is Thioredoxin-A, wherein the loop structure can be inserted within the reducing active site. Peptide aptamers can be generated/selected from various types of libraries, such as phage display, mRNA display, ribosome display, bacterial display and yeast display libraries.

As used herein, “specifically binds” refers to an association or union of a binding domain, or an affinity reagent (e.g., fusion protein) containing the binding domain, to a target molecule (e.g., CD45 or radioactive ligand) with an affinity or Kₐ (i.e., an equilibrium association constant of a particular binding interaction with units of 1/M) equal to or greater than 10⁵ M⁻¹, while not significantly associating or uniting with any other molecules or components in a sample. Binding domains can be classified as “high affinity” binding domains or “low affinity” binding domains. “High affinity” binding domains refer to those binding domains with a Kₐ of at least 10⁷ M⁻¹, at least 10⁸ M⁻¹, at least 10⁹ M⁻¹, at least 10¹⁰ M⁻¹, at least 10¹¹ M⁻¹, at least 10¹² M⁻¹, or at least 10¹³ M⁻¹. “Low affinity” binding domains refer to those binding domains with a Kₐ of up to 10⁷ M⁻¹, up to 10⁶ M⁻¹, up to 10⁵ M⁻¹. Alternatively, affinity can be defined as an equilibrium dissociation constant (Kd) of a particular binding interaction with units of M (e.g., 10⁻⁵ M to 10⁻¹³ M). In certain embodiments, a binding domain may have “enhanced affinity,” which refers to a selected or engineered binding domain with stronger binding to a target antigen than a wild type (or parent) binding domain. For example, enhanced affinity may be due to a Kₐ (equilibrium association constant) for the target antigen that is higher than the wild type binding domain, or due to a K_{d} (dissociation constant) for the target antigen that is less 10 than that of the wild type binding domain, or due to an off-rate (K_{off}) for the target antigen that is less than that of the wild type binding domain. A variety of assays are known for identifying binding domains of the present disclosure that specifically bind a particular target, as well as determining binding domain affinities, such as Western blot, ELISA, and Biacore® analysis (see also, e.g., Scatchard et al., Ann. N.Y. Acad. Sci. 51:660, 1949; and U.S. Pat. Nos. 5,283,173, 5,468,614, or the equivalent).

In some embodiments, the CD45 cancer antigen to which the first binding domain specifically binds comprises the extracellular domain of CD45. The CD45 can be from any mammal, such as mouse, rat, dog, cat, primate (including human). In some embodiments, the CD45 cancer antigen is or comprises the extracellular domain of CD45. A representative amino acid sequence for human CD45 is provided by UniProtKB/Swiss-Prot:P08575, incorporated herein by reference and set forth herein as SEQ ID NO:2, and is encoded by the nucleic acid sequence set forth in Genbank Accession No., incorporated herein by reference and set forth herein as SEQ ID NO:1. Amino acids 156-1811 of the full protein represent the extracellular domain.

The radioactive ligand to which the second binding domain specifically binds can comprise a radioactive moiety and a complexing agent. The radioactive moiety can be any moiety that, for example, is known to be effective in radiotherapy for cancer. The radioactive moiety can be a moiety that emits alpha particles, beta particles, or gamma particles. Yttrium, lutetium, astatine, iodine thorium, and actinium are illustrative and non-limiting examples of appropriate radioactive moieties. In some embodiments, the radioactive ligand also comprises a complexing agent that maintains the radioactive moiety. Appropriate complexing agents can be specifically selected for compatibility with the selected radioactive moiety. Illustrative, non-limiting examples of complexing agents include DOTA, DOTATE, and decaboron B10.

Exemplary affinity reagents (e.g., fusion proteins) with first domains that exhibit specific CD45 binding are described below. Sequences of the exemplary binding domains and encoding polynucleotides are also provided below. In some embodiments, the first binding domain comprises an amino acid sequence with at least 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to the domains disclosed herein that specifically bind to CD45.

In specific embodiments, the radioactive ligand to which the second binding domain specifically binds is or comprises yttrium-DOTA (Y-DOTA), lutetium-DOTA (Lu-DOTA), yttrium-DOTATE, lutetium-DOTATE, astatine-B10, iodine-B10, and the like.

Exemplary affinity reagents (e.g., fusion proteins) with domains that exhibit specific binding to a radioactive ligand (e.g., yttrium-DOTA) are described below. Sequences of the exemplary binding domains and encoding polynucleotides are also provided below. In some embodiments, the second binding domain comprises an amino acid sequence with at least 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to the domains disclosed herein that specifically bind to a radioactive ligand (e.g., yttrium-DOTA).

Exemplary Configurations

The bispecific affinity reagent can comprise one or more first binding domains, as described herein and one or more second binding domains in any arrangement or configuration that permits simultaneous binding of the CD45 cancer antigen and the radioactive ligand. The (one or more) binding domains can exist in the same polypeptide molecule, such as in a fusion protein, or can exist in multiple initial polypeptides that are chemically linked.

Exemplary configurations are described below. However, these are non-limiting and other configurations are encompassed by the present disclosure.

In some embodiments, the first binding domain and the second binding domain are separated by a hinge region. As used herein, a “hinge region” refers to a region that provides sufficient space and flexibility between the first and second binding domains to facilitate the binding of each to their specific antigens without mutual interference. In some embodiments, the hinge region can comprise (a) an immunoglobulin hinge sequence (made up of, for example, upper and core regions) or a functional fragment or variant thereof, (b) a type II C-lectin interdomain (stalk) region or a functional fragment or variant thereof, or (c) a cluster of differentiation (CD) molecule stalk region or a functional variant thereof. In some embodiments, the immunoglobulin hinge region can be a naturally occurring upper and middle hinge amino acid sequences interposed between and connecting the CH₁ and CH₂ domains (for IgG, IgA, and IgD) or interposed between and connecting the CH₁ and CH₃ domains (for IgE and IgM) found in the heavy chain of an antibody. In certain embodiments, a hinge region is human, and in particular embodiments, comprises a human IgG hinge region. In some embodiments, the IgG hinge region is a human IgG1 Fc hinge.

In some embodiments, hinge regions associated with the first and second binding domains, respectively, provides for a disulfide bond that chemically joins together the first and second binding domains into a single affinity reagent, such as a Fab₂.

In some embodiments, one or both of the first binding domain and the second binding domain comprises a variable light chain domain and variable heavy chain domain. The variable light chain domain and variable heavy chain domain can be separated by a linker domain. The linker domain can be a five to about 35 amino acid sequence that connects the heavy chain immunoglobulin variable region to the light chain immunoglobulin variable region. In such embodiments, the entire affinity reagent can be a single fusion protein without requiring additional chemical linkages to combine binding domains.

As indicated above, the one or both of the first binding domain and the second binding domain can be an scFv. In some configurations, the bispecific affinity reagent (also referred to as “fusion protein”) is a divalent scFv, wherein each of the first binding domain and the second binding domain is an scFv, and wherein the first binding domain and a second binding domain are joined by a linker peptide. In other configurations, the bispecific fusion protein is a tandem di-valent scFv, a diabody, or a bivalent minibody.

As indicated above, the bispecific affinity reagent (e.g., fusion protein) can comprise an additional binding domain that specifically binds to a different or additional target antigen. In some embodiments, the affinity reagent is a fusion protein characterized as a trispecific Fab₃ or triabody. In some embodiments, the additional binding domain (e.g., third binding domain) binds to a radioactive ligand. Thus, in some embodiments, the second binding domain specifically binds a first radioactive ligand and the third binding domain specifically binds a second radioactive ligand. The radioactive ligands are described above. The first radioactive ligand and the second radioactive ligand can be the same or different. In some embodiments, the radioactive ligand and the second radioactive ligand are different. In some exemplary embodiments, the first radioactive ligand comprises an alpha emitting radioactive moiety and the second radioactive ligand comprises a beta emitting radioactive moiety. In other embodiments, the first radioactive ligand comprises an alpha emitting radioactive moiety and the second radioactive ligand comprises a gamma emitting radioactive moiety. In yet other embodiments, the first radioactive ligand comprises a beta emitting radioactive moiety and the second radioactive ligand comprises a gamma emitting radioactive moiety.

Related Reagents

In additional aspects, the disclosure provides a nucleic acid comprising a sequence that encodes the bispecific affinity reagent (e.g., fusion protein) described herein, vectors comprising the nucleic acid, and cells comprising the nucleic acid and/or vector.

The vector can further comprise a promoter sequence operatively linked to the nucleic acid as appropriate for the intended host cell to promote expression of the bispecific fusion protein.

Methods

The disclosed bispecific affinity reagents (e.g., fusion proteins) are useful for specifically pre-targeting a cell for radioimmunotherapy that ultimately reduces off-target toxicity. Accordingly, in another aspect the disclosure provides a method of treating a malignancy associated with elevated expression of CD45 in a subject. The method comprises administering to the subject a therapeutically effective amount of the bispecific affinity reagent (e.g., fusion protein), as described herein, and then administering a therapeutically effective amount of a radioactive ligand, as described herein.

As used herein, the term “treat” refers to medical management of a disease, disorder, or condition of a subject (e.g., a human or non-human mammal, such as a primate, horse, dog, mouse, rat, and the like). For example, an appropriate dose or treatment regimen comprising bispecific fusion protein that bind CD45 in combination administration of a corresponding radioactive ligand, such as in a PRIT regimen, is administered to elicit a therapeutic or prophylactic benefit. Therapeutic or prophylactic/preventive benefit includes improved clinical outcome; lessening or alleviation of symptoms associated with a disease; decreased occurrence of symptoms; improved quality of life; longer disease-free status; diminishment of extent of disease, stabilization of disease state; delay of disease progression; remission; survival; prolonged survival; or any combination thereof. For example, the replication rate of a proportion of targeted CD45 expressing cells is slowed or stopped. In other outcomes, a proportion of targeted CD45 expressing cells is killed.

In some embodiments, the method further comprising administering a clearing agent (CA) after administering the bispecific affinity reagent (e.g., fusion protein) and prior to administering the therapeutically effective amount of the radioactive ligand to accelerate the clearance of any unbound bispecific affinity reagent (i.e., fusion protein) from the subject's bloodstream. The reduction of circulating (i.e., unbound) bispecific affinity reagent will reduce the likelihood that the radioactive moiety will bind to bispecific affinity reagent (i.e., fusion protein) that is not bound to CD45. This ultimately leads to a reduction in any off-target toxicity.

In some embodiments, the clearing agent is or comprises DOTAY, dextran-DOTAY, or NGB (biotinylated N-acetyl-galactosamine).

The malignancy is any malignancy characterized by an abnormal elevation in CD45 expression on the surface of the transformed cells. In some embodiments, the malignancy is a hematological malignancy. The malignancy is a T cell malignancy or a B cell malignancy. The hematological malignancy can be any leukemia, myeloma, or lymphoma. In some embodiments, the leukemia is characterized as acute lymphocytic leukemia (ALL), myelodysplastic syndromes with excess blasts, acute myelogenous leukemia (AML), chronic lymphocytic leukemia (CLL), or chronic myelogenous leukemia (CML).

In another aspect, the disclosure provides a method of producing the bispecific affinity reagent (e.g., fusion protein) as described herein. The method comprises causing the expression of the nucleic acid encoding the bispecific affinity reagent (e.g., fusion protein) in a host cell transformed therewith to promote production of the bispecific affinity reagent (e.g., fusion protein). The bispecific affinity reagent (e.g., fusion protein) can be recovered (i.e., isolated and/or purified) according to techniques known in the art.

### Additional Definitions

Unless specifically defined herein, all terms used herein have the same meaning as they would to one skilled in the art of the present invention. Practitioners are particularly directed to Sambrook J., et al. (eds.) Molecular Cloning: A Laboratory Manual, 3rd ed., Cold Spring Harbor Press, Plainsview, N.Y. (2001); Ausubel, F. M., et al. (eds.), Current Protocols in Molecular Biology, John Wiley & Sons, New York (2010); and Coligan, J. E., et al. (eds.), Current Protocols in Immunology, John Wiley & Sons, New York (2010) for definitions and terms of art.

The use of the term “or” in the claims is used to mean “and/or” unless explicitly indicated to refer to alternatives only or the alternatives are mutually exclusive, although the disclosure supports a definition that refers to only alternatives and “and/or.”

Following long-standing patent law, the words “a” and “an,” when used in conjunction with the word “comprising” in the claims or specification, denotes one or more, unless specifically noted.

Unless the context clearly requires otherwise, throughout the description and the claims, the words “comprise,” “comprising,” and the like, are to be construed in an inclusive sense as opposed to an exclusive or exhaustive sense; that is to indicate, in the sense of “including, but not limited to.” Words using the singular or plural number also include the plural and singular number, respectively. Additionally, the words “herein,” “above,” and “below,” and words of similar import, when used in this application, shall refer to this application as a whole and not to any particular portions of the application. Unless stated otherwise, the term “about” implies minor variation around the stated value of no more than 10% (above or below), 9% (above or below), 8% (above or below), 7% (above or below), 6% (above or below), 5% (above or below), 4% (above or below), 3% (above or below), 2% (above or below), or 1% (above or below).

Disclosed are materials and compositions that can be used for, can be used in conjunction with, can be used in preparation for, or are products of the disclosed methods and compositions. It is understood that, when combinations, subsets, interactions, groups, etc., of these materials are disclosed, each of various individual and collective combinations is specifically contemplated, even though specific reference to each and every single combination and permutation of these compounds may not be explicitly disclosed. This concept applies to all aspects of this disclosure including, but not limited to, steps in the described methods. Thus, specific elements of any foregoing embodiments can be combined or substituted for elements in other embodiments. For example, if there are a variety of additional steps that can be performed, it is understood that each of these additional steps can be performed with any specific method steps or combination of method steps of the disclosed methods, and that each such combination or subset of combinations is specifically contemplated and should be considered disclosed. Additionally, it is understood that the embodiments described herein can be implemented using any suitable material such as those described elsewhere herein or as known in the art.

Publications cited herein and the subject matter for which they are cited are hereby specifically incorporated by reference in their entireties.

The following describes the design and development of illustrative embodiments of an exemplary bispecific fusion protein of the present disclosure, and their use in two-step approach to pre-targeted radioimmunotherapy (PRIT) for malignancies that express CD45 surface antigen. The described strategy presents advantageous alternatives to the existing streptavidin-biotin PRIT approaches targeting CD45+ malignancies.

### Results

### Initial Proof-of-Concept Validation in Murine Leukemia Model

Production and Functional Assessment of 30F11-Fc-C825

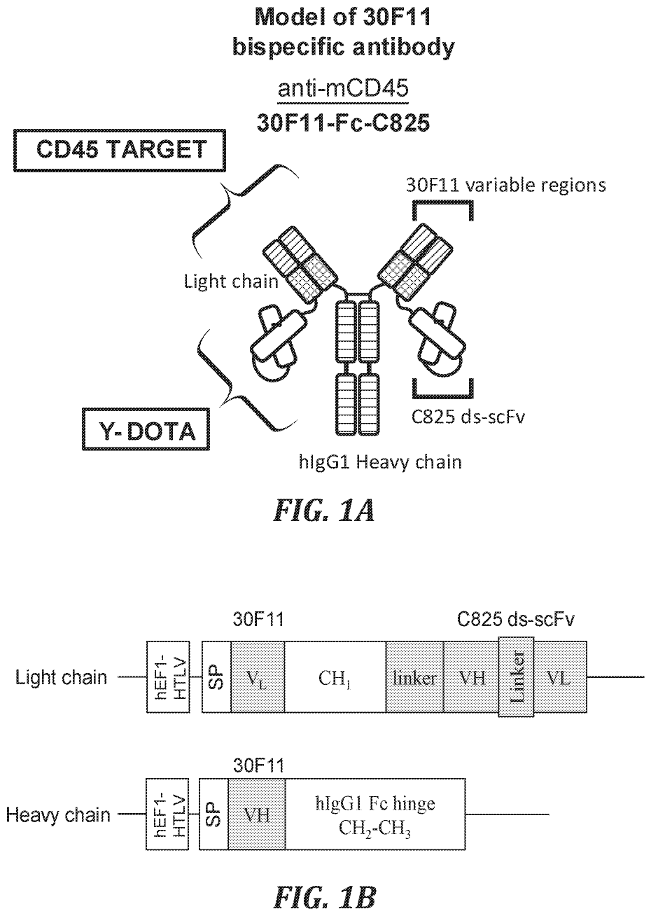

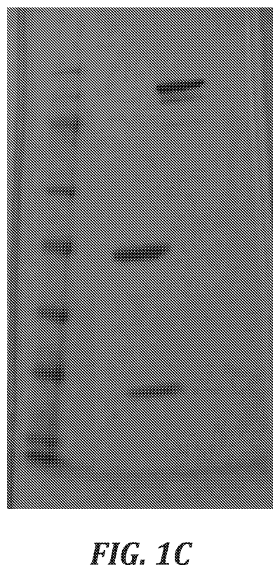

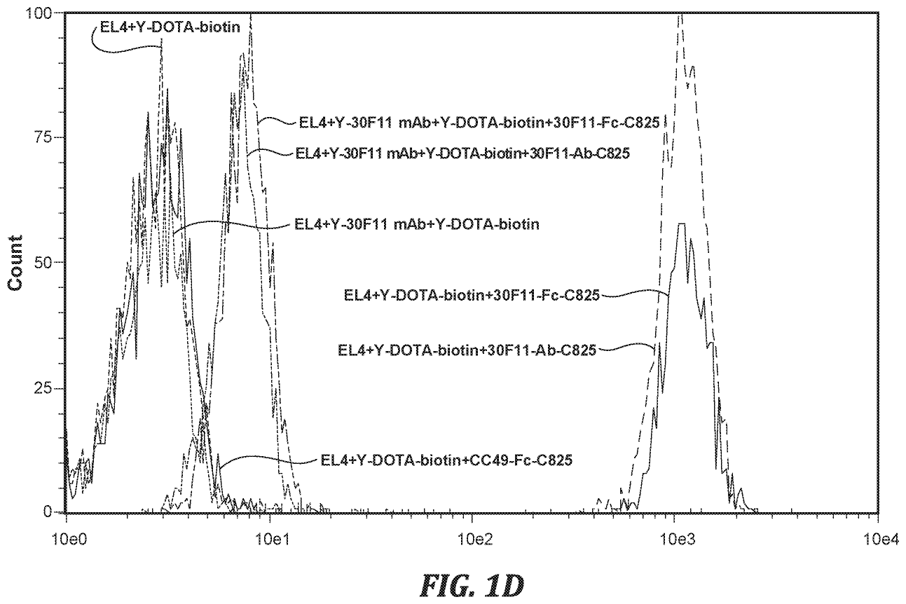

A bispecific antibody (30F11-Fc-C825), recognizing murine CD45 and Y-DOTA (FIG. 1A), was produced by cloning the regions coding for the V_{L} and V_{H} fragments of the yttrium-DOTA (Y-DOTA) capturing C825 disfulfide-stabilized scFv9ds-scFv) gene onto the light chain of the parent anti-mCD45 antibody 30F11. The vectors carrying the light or heavy chains (FIG. 1B) were co-transfected into HEK293T cells, and the supernatant was purified over a protein A column to produce bispecific antibody. Collected fractions were run over SDS-PAGE (FIG. 1C), and confirmed anticipated size of heavy and light chains. Binding functionality of 30F11-Fc-C825 bispecific antibody was verified by incubating 30F11-Fc-C825 with mCD45⁺ EL4 cells, followed by Y-DOTA, with or without 30F11 mAb blocking in excess and then assayed for SA (FIG. 1D). Secondary PE-SA Ab confirmed two 30F11-Fc-C825 batches (30F11-Fc-C825 and 30F11-Ab-C825) bound to EL4 cells, and binding was blocked by excess 30F11 mAb.

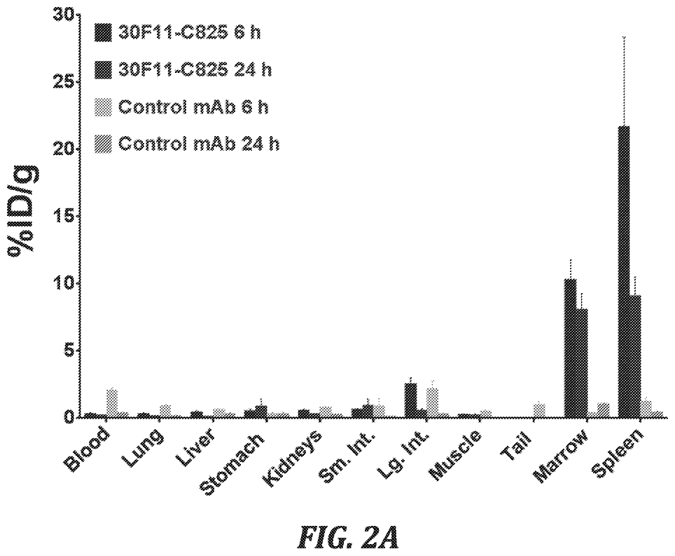

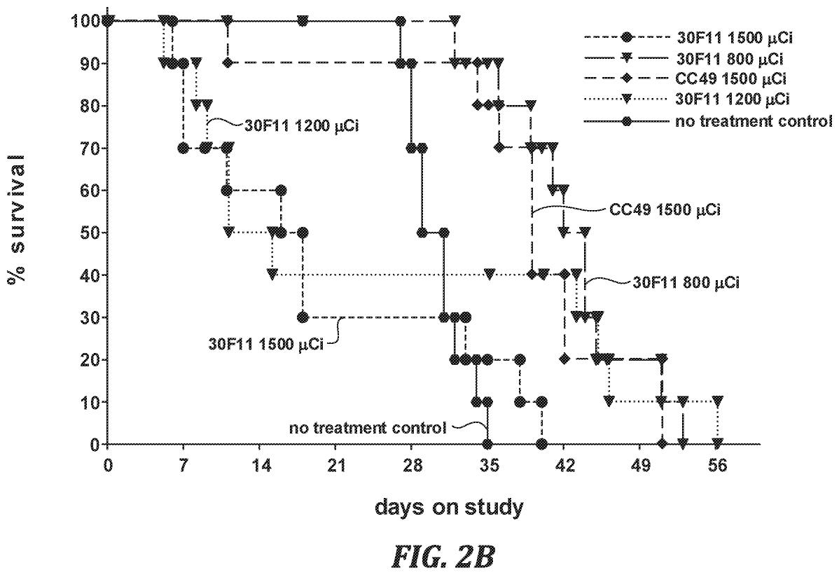

In vivo characterization and efficacy of 30F11-Fc-C825 After verification of functional binding of 30F11-Fc-C825 to both CD45⁺ cells and Y-DOTA in vitro, biodistribution was assayed over a shortened time course using mice bearing SJL tumor cells given 2 days prior (disseminated murine leukemia) to bispecific construct injections. Groups of 5 mice were given 1.4 nmol of 30F11-Fc-C825 or CC49-Fc-C825 (control Ab) as a non-targeting bispecific control, followed 23 hr later by 5 DOTAY-dextran (DYD) clearing agent. Mice were then given 50 μCi ⁹⁰Y-DOTA-biotin 24 hr after injection of first step reagents, and murine tissues were harvested at 6 and 24 hours after ⁹⁰Y-injections (FIG. 2A). CD45⁺ target tissues (spleen and marrow) exhibited significant, specific uptake by 6 hr after injection (21.7±6.7 and 10.3±1.5% ID/g, respectively) which persisted for 24 hr (9.04±1.5 and 8.08±1.2% ID/g, respectively). Non-target organs including kidneys and lungs had lower uptake (<0.5% ID/g) at this time point. There was no significant uptake in any tissue at either time point for the non-targeting bispecific antibody CC49-Fc-C825.

Having shown preferential targeting to sites of disease, efficacy of bispecific antibody constructs were then evaluated in the syngeneic, disseminated leukemia model. Groups of 10 mice were given SJL murine leukemia, followed by 1.4 nmol 30F11-Fc-C825 or non-targeting bispecific antibody CC49-Fc-C825, and 22 hr later given DYD clearing agent. Two hr after clearing agent, mice were given 800, 1200, or 1500 μCi ⁹⁰Y-DOTA-biotin (FIG. 2B). Significant treatment related toxicity was observed with mice receiving 1500 or 1200 μCi of ⁹⁰Y-DOTA-biotin. There were no long-term survivors when treated with 30F11-Fc-C825 at these radioactivity amounts, but there was a therapeutic benefit at the lower 800 μCi dose. SJL leukemia bearing mice treated with 30F11-Fc-C825 and 800 μCi of ⁹⁰Y-DOTA-biotin had median overall survival (OS) of 43 days, compared to disease control mice (OS=30 days, p<0.0001).

### Optimizing Human Leukemia Model

Production and Functional Assessment of BC8-Fc-C825

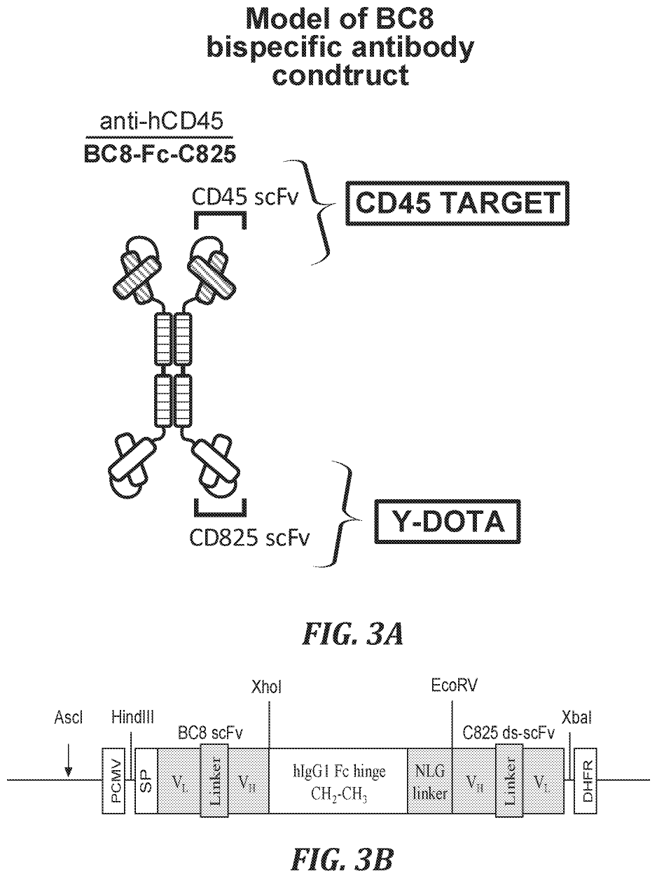

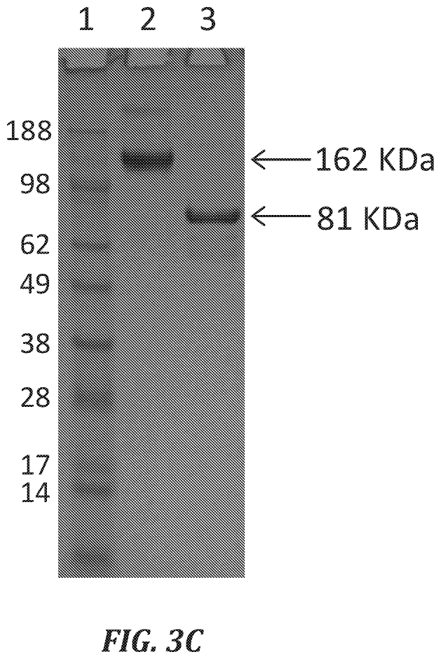

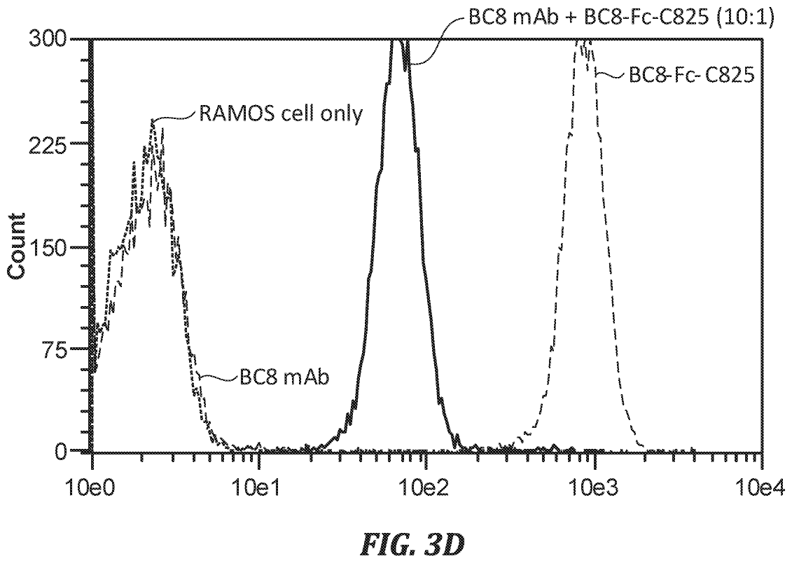

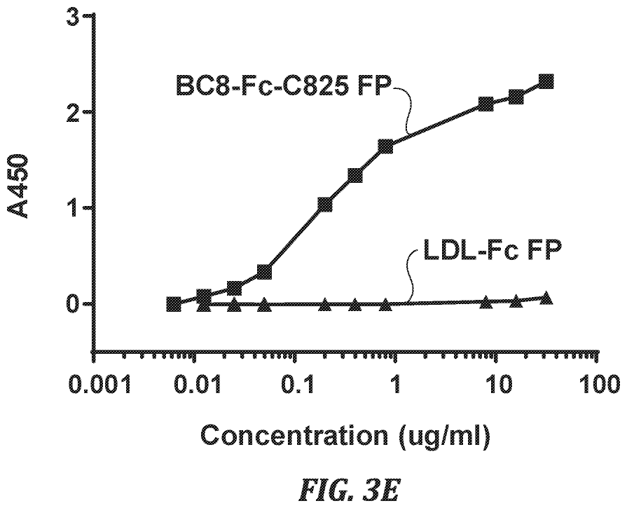

After initial proof-of-concept studies in the syngeneic murine leukemia model, the inventors designed a bispecific construct for the more clinically relevant human leukemia model. The bispecific fusion protein recognizing both human CD45 and Y-DOTA, (BC8-Fc-C825 FP), was produced by cloning the region coding for the scFv of the BC8 antibody, with the scFv of the DOTAY-specific C825 antibody (FIG. 3A). The construct (FIG. 3B) was transfected into CHO cells under methotrexate selection, and the bispecific antibody purified by protein A columns. Collected fractions were assessed via SDS-PAGE for purity of antibody produced and verification of size, estimated at 81 kDa for the monomer (FIG. 3C). Functional binding of BC8-Fc-C825 FP was assessed via flow cytometry for binding to Ramos cells, a CD45-expressing human Burkitt's Lymphoma line (FIG. 3D). Functional recognition of Y-DOTA was assayed by quantification of bound BC8-Fc-C825 serially diluted amounts of bound Y-DOTA, whereas the negative bispecific LDL-Fc-FP did not show any significant binding at any Y-DOTA concentration (FIG. 3E).

In Vivo Blood Clearance of BC8-Fc-C825

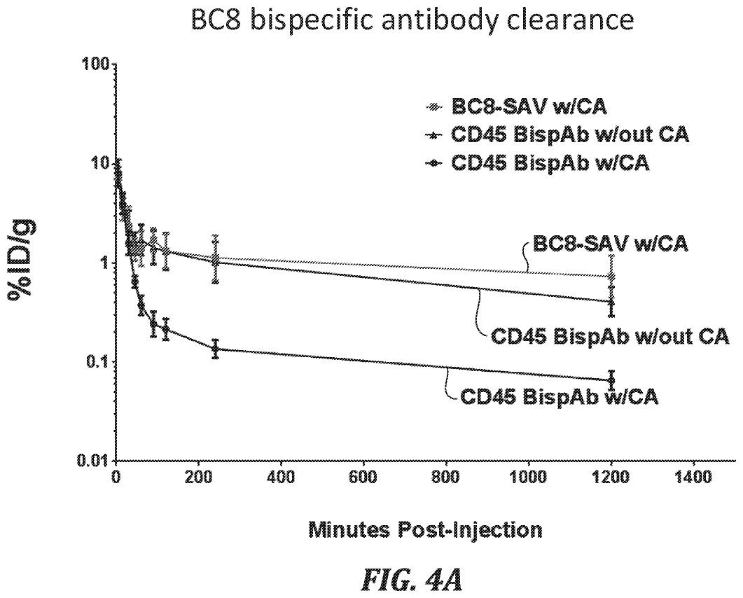

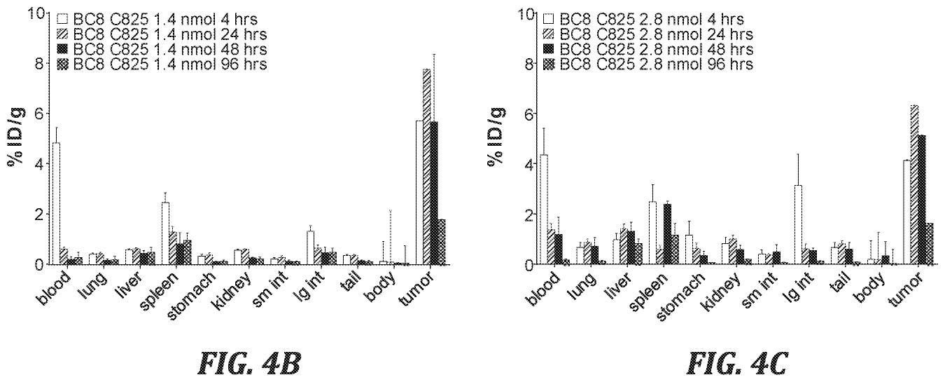

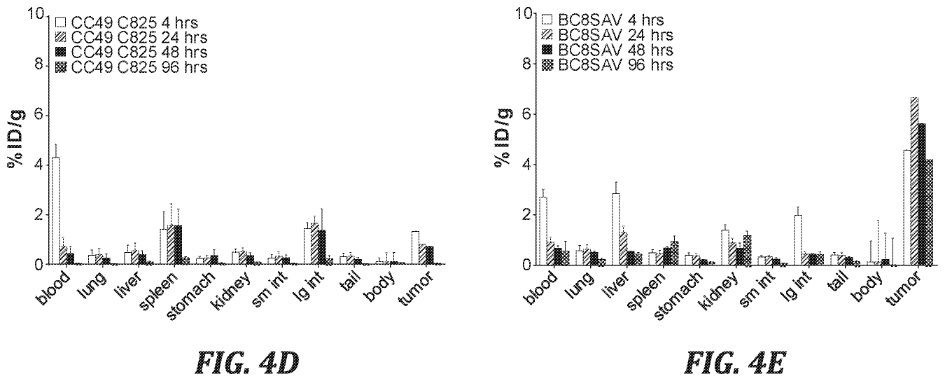

Blood clearance of the BC8-Fc-C825 bispecific antibody was compared with clearance of streptavidin-modified BC8 (BC8-SA) by the pretargeted radioimmunotherapy (PRIT) approach. Because attempts to radiolabel bispecific constructs with iodine-131 were unsuccessful, blood clearance was studied by following the radiolabeled 2^{nd} step, or ⁹⁰Y-labeled DOTA. Bispecific antibody clearance was studied by injecting 1.4 nmol of BC8-Fc-C825, followed by DOTAY-dextran clearing agent (DYD) or no DYD 23 hours later, followed by ⁹⁰Y-DOTA-biotin 24 hours after the first step reagent. SA-based PRIT treated mice were given 1.4 nmol BC8-SA and 21 h later given a sarcosyl-biotin clearing agent (CA) before infusing ⁹⁰Y-DOTA-biotin 3 hours after the first step reagent. Blood was taken from treated mice at various time points (5 min, 10 min, 15 min, 30 min, 1 h, 2 h, 4 h and 20 h) after injection of ⁹⁰Y-DOTA-biotin. Clearance of BC8-Fc-C825 without any CA injection was similar to clearance of BC8-SA using CA, as % injected dose/gram of blood (% ID/g) at 20 hr was 0.41±0.2% and 0.73±0.5% respectively, with nearly overlapping clearance curves (FIG. 4A). However, BC8-Fc-C825 given with CA had significantly faster clearance, with only 0.07±0.02% ID/g at 20 hours.

In Vivo Characterization and Efficacy of BC8-Fc-C825

Because blood clearance of circulating BC8-Fc-C825 bispecific antibody was improved with CA treatment, subsequent biodistribution and therapy studies included CA injections. Next, biodistribution and dosimetry studies with ⁹⁰Y-DOTA-biotin were pursued to compare tissue distribution of BC8-Fc-C825 bispecific antibody with BC8-SA and the non-binding control CC49-Fc-C825 bispecific antibody. Different amounts (1.4 nmol or 2.8 nmol) of bispecific antibody (BC8-Fc-C825) were injected into athymic nude mice with palpable HEL (hAML) tumor xenografts, followed 23 hr later by DYD CA to remove unbound antibody, followed after 24 hr with ⁹⁰Y-DOTA-biotin. Tissue harvesting was performed at 4, 24, 48, and 96 hr after injection of ⁹⁰Y-DOTA. Within 4 hr after ⁹⁰Y-DOTA injection, specific and robust uptake by tumor in the group receiving 1.4 nmol was seen at 5.70±0.01% ID/g, peaking at 7.75±0.02% ID/g 24 hr after injection, and persisting stably for at least 48 hr (FIG. 4B). Uptake in non-target tissues was minimal with peak uptake in the kidneys of 0.51±0.3% ID/g at 24 hr. Mice receiving 2.8 nmol (FIG. 4C) also had rapid, specific uptake peaking at 6.32±0.01% ID/g 24 hr post-injection, though non-target tissues showed slightly more non-specific uptake than the 1.4 nmol group, with peak uptake in the kidneys of 1.0±0.3% ID/g at 24 hr (FIG. 4C). CC49-C825, a non-binding bispecific antibody, showed minimal uptake in all tissues (FIG. 4D). The group receiving BC8-SA as a first step reagent prior to clearing agent and then ⁹⁰Y-DOTA-biotin showed similar rapid, robust uptake in tumor with minimal uptake in non-target tissues as the BC8-C825 groups. Peak uptake in tumor was 6.65±0.01% ID/g 24 hr after injection compared to 1.4±0.5% ID/g peak uptake in the kidneys 4 h after injection (FIG. 4E).

### Dosimetry Studies

Absorbed radiation doses to tissues were calculated for mice treated with BC8-Fc-C825 as a first step reagent followed by clearing agent and ⁹⁰Y-DOTA-biotin. This analysis showed that BC8-Fc-C825 effectively targeted the high doses to tumor, with 3.978 cGy and 3.518 cGy per μCi ⁹⁰Y injected for 1.4 nmol and 2.8 nmol of injected construct, respectively, and comparable to doses targeted with BC8-SA, 4.66 cGy per μCi ⁹⁰Y injected (Table 1). The doses achieved in tumor with the non-targeting bispecific antibody were similar to blood 0.423 in the tumor compared to 0.575 cGy/μCi ⁹⁰Y injected in blood. Absorbed doses from ⁹⁰Y showed tumor-to-normal organ ratios of 7.2 for liver, 15.8 for kidney, and 48.6 for whole body for mice receiving 1.4 nmol of BC8-C825 (Table 1). Similar values were seen in mice treated with 1.4 nmol of BC8-SAV, which resulted in tumor-to-normal-organ doses of 5.19 for liver, 7.82 for kidney, and 23.6 for total body.

TABLE 1
Absorbed radiation doses of 90Y-DOTA-biotin as the second
step agent after Fc-C825 FP Bispecific Antibody, BC8-SAV,
or non-targeting CC49-Fc-C825) (cGy/μCi injected)

| | BC8-C825 | BC8-C825 | | |
|---|---|---|---|---|
| Tissue | 1.4 nmol | 2.8 nmol | BC8-SAV | CC49-C825 |
| Blood | 0.586 | 0.817 | 0.526 | 0.575 |
| Lung | 0.112 | 0.296 | 0.240 | 0.113 |
| Liver | 0.551 | 1.476 | 0.899 | 0.390 |
| Spleen | 0.487 | 0.740 | 0.757 | 0.701 |
| Stomach | 0.068 | 0.145 | 0.089 | 0.090 |
| Kidney | 0.252 | 0.449 | 0.597 | 0.248 |
| Sm Int | 0.057 | 0.113 | 0.087 | 0.076 |
| Lg Int | 0.446 | 0.582 | 0.402 | 1.146 |
| Tail | 0.205 | 0.579 | 0.320 | 0.202 |
| Total Body | 0.0818 | 0.286 | 0.198 | 0.144 |
| Tumor | 3.978 | 3.518 | 4.666 | 0.423 |

### Therapy Studies

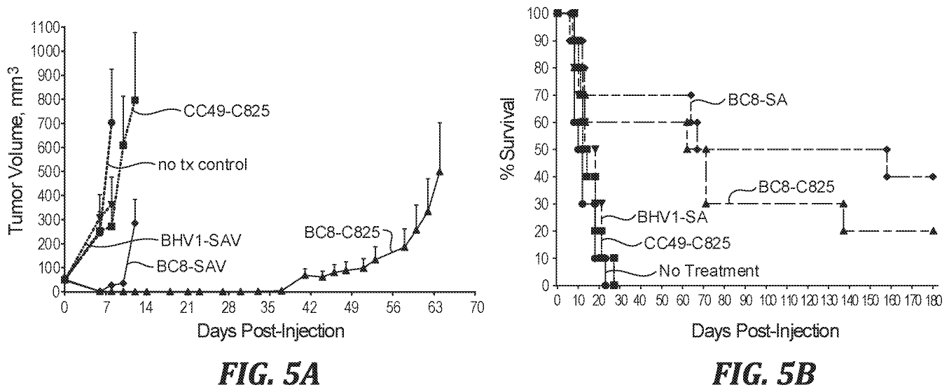

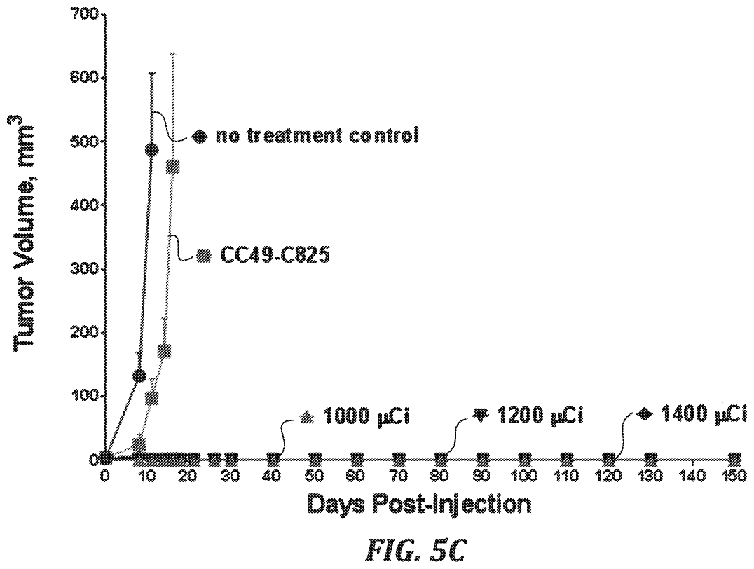

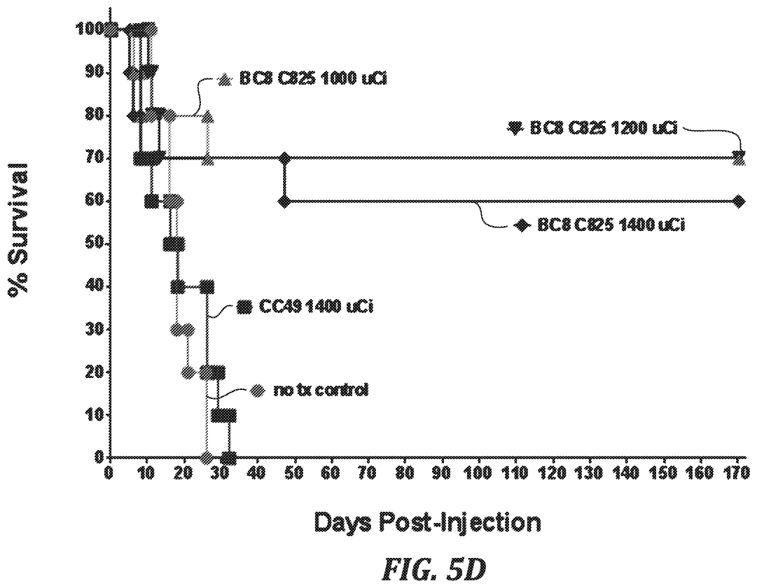

Having confirmed tumor-specific targeting with both bispecific constructs by biodistribution and dosimetry studies, this approach was evaluated for efficacy in therapy studies. Groups of 10 mice bearing palpable subcutaneous HEL xenografts were treated with 1.4 nmol of BHV1-SAV or CC49-C825 (non-binding Ab-SAV or bispecific antibody, respectively), BC8-SAV or BC8-Fc-C825 FP. The next day (21 hr later) mice were injected with a sarcosyl-biotin clearing agent (Ab-SAV groups) or a DYD (DOTAY-dextran) clearing agent. Approximately 24 hr after injection of targeting first step constructs, mice were given 1400 μCi ⁹⁰Y-DOTA-biotin. Mice were assessed for tumor volumes, body weights, and toxic effects for up to 180 d. Tumor volume curves were truncated after the first death in each group due to disparities in tumor sizes (FIG. 5A). Treatment toxicity was similar in both BC8-SAV and BC8-C825 treated groups, with 3 to 4 early deaths (out of groups of 10) in either group. Furthermore, progression-free survival (PFS) for BC8-SAV treated mice (64 days) was similar to PFS in BC8-Fc-C825 treated mice (62 days; FIG. 5B). There was no statistically significant difference in long-term survival between the groups (180 days post-injection, FIG. 5B) via Mantel-Cox test (p value 0.38) for mice treated with BC8-SA or BC8-Fc-C825. Mice receiving BC8-Fc-C825 had statistically better survival outcomes than mice receiving CC49-C825. Groups of untreated mice and those receiving CC49-Fc-C825 were all euthanized due to tumor growth by day 27 after ⁹⁰Y-DOTA-biotin injection.

Having shown efficacy with rather high ⁹⁰Y-DOTA-biotin activity, dose optimization studies were conducted. Mice bearing a subcutaneous xenograft 2 days after implantation received 1.4 nmol of BC8-Fc-C825 or CC49-Fc-C825 bispecific FP, followed 22 hr later by DYD clearing agent. Mice were given 1000, 1200, or 1400 μCi of ⁹⁰Y-DOTA-biotin 24 hr after injection of bispecific constructs. Tumor volume curves were truncated when the first mouse in its group was euthanized due to tumor size (FIG. 5C). By day 170 after ⁹⁰Y-DOTA-biotin injection, no mice had been euthanized due to tumor growth in any of the BC8-Fc-C825 treatment groups. Although each BC8-C825 treatment group had early deaths due to treatment-related toxicity, 6 mice in the 1400 μCi group and 7 mice in the 1200 and 1000 μCi groups survived disease-free at least 170 days after ⁹⁰Y-DOTA-biotin injection (FIG. 5D). Non-treated diseased mice and non-targeting FP CC49-C825-treated mice were all euthanized due to tumor growth by day 26 or 32, respectively.

### DISCUSSION

The studies described herein demonstrate that bispecific antibody-based fusion protein constructs targeting either human or murine CD45 and DOTAY are effective therapeutic agents in preclinical leukemia models. Biodistribution studies showed that targeting of both murine and human bispecific constructs exhibited the highest uptake with their corresponding CD45+ target, with highest uptake of human CD45 specific BC8-Fc-C825 in the HEL xenografts, and highest uptake of murine CD45 specific 30F11-Fc-C825 in the spleen and marrow of mice with disseminated murine leukemia. Furthermore, dosimetry calculations for BC8-Fc-C825 also showed the highest absorbed doses for HEL xenograft tumors, as would be expected for specific targeting. Lastly, BC8-Fc-C825 was therapeutically as effective as the biotin-streptavidin approach in HEL bearing mice (FIG. 4B), with both constructs yielding long term survivors. This is consistent with prior work showing efficacy with sparing of normal organs with other SA-biotin pretargeting approaches.

The disclosed bispecific fusion protein constructs increase the repertoire of bispecific reagents that target Y-DOTA to treat hematologic malignancies with CD45 expression. Moreover, this targeted therapeutic strategy delivering radiation to hematologic malignancies is less likely to be vulnerable to clonal resistance seen with other narrowly targeted approaches for particular molecular mutations or targets, such as BTK or b1c2 inhibitors, without sustained evolutionary pressure on clonal subsets.

When compared with other therapeutic approaches to treat leukemia, bispecific Ab pretargeting of CD45 and Y-DOTA appears to be as effective as other approaches to treat AML in preclinical models. Not surprisingly, CD33 has been an AML target for many of these, with the majority of bispecific antibodies redirecting T-cell activity to CD33+ leukemia cells. The first bispecific T-cell engaging construct (AMG 330) has been shown to decrease the growth of AML xenograft tumors in humanized mice and to improve overall survival, where about 50% of mice treated at the highest dose were long term survivors. This is not surprising as the objective of the construct is to approximate the target cell with powerful cytotoxic cells that can activate the immune system for tumor clearance. However, clinical approaches using T-cell engaging constructs have been associated with clinically significant cytokine release syndrome and immune activation that may have contributed to suspension of the AMG 330 clinical trial (NCT02520427). The bispecific constructs used in the presently described study targeting CD45 and DOTAY do not directly approximate T-cells and should have minimal effects on activating the immune system and inducing cytokine storm. Furthermore, when the BC8 Ab has been used in clinical trials, infusion-related reactions are sometimes observed, likely because of the murine origins of the Ab, and can readily be reversed with supportive measures and complete the infusion the same day.

Other CD33 targeting bispecific approaches have redirected the immune system beyond cytotoxic T-cells, to the innate immune system such as NK cells, by also targeting CD16. A humanized scFv of CD16 and CD33 linked together as a BiKE, was able to activate NK cells and lyse CD33+ AML cells in vitro. This approach has been advanced further with tri-specific killer engagers (TriKEs) by the addition of cytokine sequences such as IL-15 to promote persistence and expansion of NK cells. An IL-15 TriKE showed improved efficacy compared to the BiKE in a murine AML xenograft model that incorporated human NK cells. While promising, these bispecific approaches require functional immune cells to eradicate the tumor, but many patients with hematologic malignancies may have an immune system without optimal immune compartments. In contrast, the presently described bispecific constructs targeting Y-DOTA do not rely solely on host immune cells to eradicate the tumor, and instead exploit the increased radiosensitivity of hematologic malignancies.

An alternative option to improve efficacy in the disseminated model could be to increase radioactivity used. However, this may be difficult to achieve given the toxicity observed in the mice treated in the higher dose groups. Although no formal toxicity studies were part of the investigations described herein, myelotoxicity was most likely given the subcutaneous hemorrhaging, and anemic blood/organs observed on necropsy of the mice dying within 2 weeks of treatment. Indeed myelotoxicity was also appreciated when utilizing higher doses of radioactivity to target solid organ malignancies, via both SA-biotin PRIT and bispecific approaches. In addition, transient myelotoxicity has been described targeting the marrow with directly labeled ⁹⁰Y-anti-CD45 RIT that was more profound than when ⁹⁰Y was used in anti-CD20 PRIT in murine lymphoma xenografts.

In summary, the presently described antibody-based bispecific fusion proteins address the immunogenicity and endogenous biotin concerns of the SA-biotin pretargeted RIT approaches. The studies described herein confirm the therapeutic efficacy of bispecific fusion proteins targeting CD45 and Y-DOTA to treat CD45+ malignancies, such as leukemia in preclinical leukemia models. Targeting was specific for CD45+ target organs, with minimal uptake in non-target organs. While BC8-Fc-C825 was as effective as the SA-biotin approach with BC8 in therapy studies, targeting murine CD45 did not yield any long-term survivors likely from the lack of cross fire effect in the disseminated setting. These studies establish the promise of additional bispecific fusion protein approaches to deliver beta or alpha-emitters in PRIT as a means to improve efficacy in disseminated disease settings.

### Examples

The following provides exemplary methods and materials for construction and utilization of illustrative embodiments of the bispecific fusion protein as disclosed herein.

Construction of a Bispecific BC8-Fc-C825 Fusion Gene

A bispecific fusion gene was constructed as described with a parallel CD20 system, using the PCR-obtained sequences for the BC8 scFv targeting human CD45 (see supplemental methods below for details) generating the BC8-Fc-C825 bispecific anti-CD45 and anti-Y-DOTA fusion gene.

Expression and Production of the BC8-Fc-C825 Fusion Protein

CHO cells expressing bispecific fusion protein were subcloned to generate a line that stably expresses BC8-Fc-C825. This line was then used to express batches of fusion protein, which was isolated from supernatants via protein A column (Repligen Bioprocessing 10-2500-03) and filter-sterilized before further characterization (see supplemental methods for details).

Production of 30F11-Fc-C825

HEK293T cells were maintained in Freestyle 293 Expression Medium with 1% pen-strep (Invitrogen, 12338 and 15140). Pfuse plasmid constructs (Invivogen, pfuse-hchal and pfuse2-hclk) carrying the heavy and light chain genes for 30F11-Fc-C825 were grown in DH5a cells (Invitrogen 18258012) under selection and prepared using an endo-free maxi-prep kit (Qiagen, 12162). Constructs were co-transfected into HEK293T (ATCC, CRL-3216) cells using 293fectin (Invitrogen, 12347). Three days post-transfection, selection was added. They were cultured until viability fell below 60%, then supernatants were collected and filtered through a 0.45 μm vacuum filtration apparatus (Millipore, schvu02re). Filtrates were loaded onto a Protein A column (GE Healthcare, 45500123) and eluted in citrate buffer at pH 3.2. Fractions were run on a 4-12% Bis-Tris SDS PAGE gel (Invitrogen, NP0335) to assess integrity of assembled antibody. Retained fractions were pooled, then dialyzed overnight in a SlideALyzer G2 cassette with a MWCO of 20,000 (Pierce, 87735) against PBS (Gibco, 14190), filtered through a 0.22 μm syringe filter unit (Millipore, SLGP033RS), then stored at 4° C.

Mice and Cell Lines

Female athymic nude Foxnl^{nu} mice, 7-10 weeks of age (Envigo athymic nude) were used in HEL (human AML) flank xenograft studies. Female B6SJLF1/J mice, 8-12 weeks of age (Jackson, 100012) were used in SJL (murine AML) disseminated syngeneic studies. Mice were housed under protocols approved by the Fred Hutchinson Cancer Research Center Institutional Animal Care and Use Committee HEL cells were purchased from ATCC (TIB-180); SJL cells were obtained as previously described, {Resnitzky, 1985} and maintained by serial transplantation in host mice.

Radiolabeling and Synthesis of Clearing Agents

DOTA-biotin was produced and radiolabeled as described. {Pagel, 2003} Radiolabeled product was assessed for radiochemical purity via an avidin-bead assay (Sigma) and injected at >90% purity. A biotinylated sarcosyl NAGB clearing agent used for Ab-SAV groups was synthesized as described. DOTAY-dextran clearing agent used for bispecific antibody groups was synthesized as described.

Blood Clearance Studies

For clearance of radiolabeled second step groups of 5 nude mice bearing HEL flank tumors were injected intravenously with 1.4 nmol of BC8-Fc-C825 or BC8-SA followed by 5 μg DOTAY-dextran or 50 μg NAGB clearing agent 22 hrs later. Two hours after clearing agent, 50 μCi of ⁹⁰Y-DOTA-biotin was injected. Mice were bled via the retro-orbital space after injection of ⁹⁰Y-DOTA-biotin. Blood was assessed on a Packard Cobra II (Perkin Elmer) for gamma counts and % injected dose per gram (% ID/g) of each sample was calculated.

Biodistribution and Dosimetry

Groups of 5 nude mice were injected with 10×10⁶ HEL cells in the right flank and palpable tumors were allowed to grow while mice were maintained on biotin-free chow (Animal Specialties, 23979). Mice were injected intravenously with 1.4 nmol, or 2.8 nmol, BC8-Fc-C825, BC8-SA, or CC49-Fc-C825. Twenty two hr post-first step reagent injection, mice were given 5 μg DOTAY-dextran clearing agent via the intraperitoneal space to remove unbound circulating antibody, followed two hours later with 50 μCi ⁹⁰Y-DOTA-biotin. Tissue harvests were performed at 4, 24, 48, and 96 hr post-injection and tissues were assessed for gamma counts to determine the % ID/g. For biodistribution studies targeting murine CD45, mice were given 10⁵ SIL murine AML intravenously. Twenty four hr later they were given 1.4 nmol 30F11-Fc-C825 or CC49-Fc-C825 intravenously, followed 22 hr later by 5 μg DOTAY-dextran clearing agent. Two hr after clearing agent, mice were given 50 μCi ⁹⁰Y-DOTA-biotin. Tissues were harvested at 6 and 24 hr post-injection of DOTA-biotin and placed on Packard Cobra II for gamma counts. After calculating the % ID/g, radiation absorbed doses were calculated for all tissues, as described previously. The equilibrium dose constant for ⁹⁰Y is 1.99 g-cGy/uCi-hours.

Therapy Studies

Groups of 10 nude mice were given 10×10⁶ HEL cells subcutaneously on the right flank for studies targeting human CD45. In one therapy study, mice were maintained on biotin-free chow (Animal Specialties) to prevent interference of endogenous biotin with Ab-SA conjugate binding. Eight days after tumor implantation mice were given 1.4 nmol of BC8-Fc-C825, CC49-Fc-C825, BC8-SAV, or BHV1-SAV intravenously. 22 hr later mice were given 5 μg DOTAY-dextran (bispecific antibody groups) or 50 μg NAGB (Ab-SA groups) clearing agent. Two hr after clearing agent, mice were given 1200 μCi ⁹⁰Y-DOTA-biotin. In other therapy studies, groups of 10 nude mice were given 10×10⁶ HEL cells subcutaneously on the right flank, and tumors were allowed to grow for 2 days before intravenous injection of 1.4 nmol CC49-Fc-C825 or BC8-Fc-C825. Twenty two hr later mice were injected with 5 μg DOTAY-dextran clearing agent, followed 2 hr later with injection of 800, 1000, or 1200 μCi ⁹⁰Y-DOTA-biotin. In syngeneic murine CD45 studies, groups of 10 B6SJL/J mice were given 10⁵ SJL cells intravenously for therapy of disseminated leukemia. 24 hr later, they were injected with 1.4 nmol 30F11-Fc-C825 or CC49-Fc-C825 intravenously. 22 hr later, mice were given 5 μg DOTAY-dextran clearing agent, followed 2 hr later by 800, 1000, or 1200 μCi ⁹⁰Y-DOTA-biotin. Four days after DOTA-biotin, mice were given 10×10⁶ syngeneic hematopoietic stem cells. For all studies, weights and tumor volume were monitored up to 170 days post-injection.

Supplemental Materials and Methods

Construction of a Bispecific BC8-Fc-C825 Fusion Gene.

An O89-1-6 mammalian expression vector containing a bispecific 2H7-hIgG1-C825 gene under the control of the CMV promoter was constructed as described previously. A signal peptide fragment was obtained from the O89-1-6 plasmid by PCR and an anti-CD45 BC8 Vl-Vh scFv fragment was obtained by PCR from an E121-3-10 plasmid. A HindII-XhoI fragment was obtained by a splicing overlapping PCR from the signal fragment and the BC8 scFv fragment using flanking oligos YL641 (ATCAACGGGACTTTCCAAAATGTC) and YL761 (TTTGGGCTCGAGAGAGCTCACGGTGACTGAGGTTCC), followed by restriction digestion with HindIII and XhoI. The HindIII-XhoI fragment was cloned into the vector O89-1-6 digested with the same restriction enzymes resulting in a P22-1 construct generating the BC8-Fc-C825 bispecific anti-CD45 and anti-Y-DOTA fusion gene.

Isolation of Stably Expressing CHO Cell Clones.

The P22-1 plasmid DNA was prepared using an endo-free maxi preparation kit (Qiagen, Germantown, Md.). The plasmid DNA (250 μg) was linearized with Ascl at the 5′ nonessential region of a CMV promoter. The DNA was purified by phenol extraction and NaOAc/ethanol precipitation. The linearized DNA was resuspended in 400 μl of serum free tissue culture medium, Excell 302 (Sigma, St Louis, Mo.). CHO DG44 cells were kept in logarithmic growth in Excell 302 supplemented with 4 mM glutamine, 1 mM sodium pyruvate, recombinant insulin (Invitrogen, Grand Island, N.Y.) and penicillin-streptomycin including 1×HT supplement (Invitrogen). 2×10⁷ cells were harvested for each transfection and resuspended in 400 μl complete medium with HT supplement. The Ascl-linearized DNA solution was added to the CHO cells in a total volume of 0.8 ml and transferred into a cuvette (4 mm gap) for electroporation using Gene Pulser Xcell (BioRad, Hercules, Calif.) at 280 volts, 950 μFarads. The transfected cells were incubated in a non-selective media overnight and plated in 96-well flat bottom plates (Costar) at various dilutions ranging from 500 cells/well to 4000 cells/well in the complete medium containing 50 nM methotrexate (Sigma) without HT supplement. The plated cells were fed every five days with the same selective media until the colonies appeared. The culture supernatants from master wells were screened for expression of -Ig fusion protein using an IgG sandwich ELISA, described briefly as follows. The NUNC plates (Fisher Scientific, Pittsburgh, Pa.) were coated overnight at 4° C. with 2 μg/ml goat anti-human IgG (Jackson Immunoresearch, West Grove, Pa.) in 70 μl of PBS. The plates were blocked at room temperature with 200 μl of 2% BSA/PBS for 1-2 hours. After washing serial dilutions of culture supernatants were added and incubated overnight at 4° C. The plates were washed three times in PBS/0.05% Tween 20 buffer and incubated with 100 μl/well horseradish peroxidase conjugated F (ab′2) goat anti-human IgG (Jackson Immunoresearch) at a 1:7500 dilution in PBS/0.5% BSA for 1 hour at room temperature. The plates were washed four times with the PBS-Tween 20 buffer and the binding detected with SureBlue, TMB substrate (KPL Labs, Gaithersburg, Md.). The reactions were stopped by adding 100 μl of 1N HCL and the plates were read at 450 nm on a Synergy 2 plate reader (Biotek, Winooski, Vt.). The clones with the highest expression of the bispecific fusion protein were expanded into T25 and T75 flasks for further amplification and treated progressively with MTX complete culture medium ranging from 50 nM to 500 nM. The supernatant from these cells were measured for -Ig fusion protein expression using the same sandwich ELISA. All bispecific-fusion-protein-expressing cells were expanded and preserved cryogenically.

Expression and Production of the BC8-Fc-C825 Fusion Protein.

One of highest expressing clones, 7G4/400, was thawed from 1 frozen vial (10⁷ cells) and washed with RPMI-10% FBS medium. The cells were resuspended in 10 ml Excell 302 complete medium containing 50 nM MTX in T25 flask and incubated at 37° C., 5% CO₂ overnight. On the next day, the cells were pelleted and transferred into a T75 flask containing 30 ml of complete medium with 400 nM MTX supplement. The cultures were expanded and passaged every 3-4 days in T175 flasks with 100 ml complete culture medium containing 400 nM MTX per flask. The expanded cells were diluted into 40 T175 flasks with 100 ml per flask complete culture medium plus 400 nM MTX at a density of 1×10⁵ cells/ml. The cultures were continued to be incubated for 13-14 days. The supernatants were collected and filtered through 0.22 μm Millipore PES membrane filter units. The pH of the supernatant was adjusted to 8.0 with 1M Na₂CO₃ solution and sodium azide was added to a final concentration of 0.1%. The conditioned supernatant was loaded on a 15-ml protein A-agarose (IPA 400HC crosslinked agarose) column (RepliGen, Waltham, Mass.) and washed with a 10-column volume of PBS (˜150 ml) by gravity flow. The BC8-Fc-C825 fusion protein was eluted with 0.1M sodium citrate buffer at pH 3.6. The concentration of the eluted protein in each fraction (˜1 ml size) was measured at 280 nm using a Nanodrop spectrometer. The fractions containing the fusion protein were pooled and dialyzed against PBS overnight at room temperature. The fusion protein was sterile filtered through 0.1 μm PVDF filter unit and stored at 4° C.

While illustrative embodiments have been illustrated and described, it will be appreciated that various changes can be made therein without departing from the spirit and scope of the invention.

The embodiments of the invention in which an exclusive property or privilege is claimed are defined as follows:

1. A bispecific affinity reagent, comprising:
a first binding domain that specifically binds to a CD45 cancer antigen, and
a second binding domain that specifically binds to a radioactive ligand.

2. The bispecific affinity reagent of claim 1, wherein the first binding domain and the second binding domain are separated by a hinge region.

3. The bispecific affinity reagent of claim 2, wherein the hinge region is an IgG1 Fc hinge.

4. The bispecific affinity reagent of claim 1, wherein one or both of the first binding domain and a second binding domain comprises a variable light chain domain and variable heavy chain domain.

5. The bispecific affinity reagent of claim 4, wherein the variable light and variable heavy chain domain are separated by a linker domain.

6. The bispecific affinity reagent of claim 1, wherein one or both of the first binding domain and a second binding domain is an antibody, a functional antibody fragment, or a functional antibody derivative.

7. The bispecific affinity reagent of claim 6, wherein one or both of the first binding domain and a second binding domain is an scFv.

8. The bispecific affinity reagent of claim 7, wherein the bispecific affinity reagent is a di-valent scFv, wherein each of the first binding domain and the second binding domain is an scFv, and wherein the first binding domain and a second binding domain are joined by a linker peptide.

9. The bispecific affinity reagent of claim 8, wherein the bispecific affinity reagent is a tandem di-valent scFv, diabody, or a trispecific Fab₃.

10. The bispecific affinity reagent of claim 9, wherein the trispecific Fab₃ further comprises a third binding domain that specifically binds to a radioactive ligand.

11. The bispecific affinity reagent of claim 10, wherein the third binding domain that specifically binds to a radioactive ligand is different than the radioactive ligand to which the second binding domain specifically binds.

12. The bispecific affinity reagent of claim 1, wherein the radioactive ligand comprises a radioactive moiety that emits alpha particles or beta particles.

13. The bispecific affinity reagent of claim 12, wherein the radioactive moiety is selected from yttrium, lutetium, astatine, iodine thorium, and actinium.

14. The bispecific affinity reagent of claim 12, wherein the radioactive ligand further comprises a complexing agent.

15. The bispecific affinity reagent of claim 14, wherein the complexing agent comprises DOTA, DOTATE, and decaboron B10.

16. The bispecific affinity reagent of claim 1, wherein the radioactive ligand is or comprises yttrium-DOTA (Y-DOTA), or lutetium-DOTA (Lu-DOTA), or yttrium-DOTATE, or lutetium-DOTATE, or astatine-B10, or iodine-B10.

17. A method of treating a malignancy associated with elevated expression of CD45 in a subject, comprising:
administering to the subject a therapeutically effective amount of the bispecific affinity reagent of any one of claims 1-16; and
administering a therapeutically effective amount of a radioactive ligand.

18. The method of claim 17, further comprising administering a clearing agent after administering the bispecific affinity reagent and prior to administering the therapeutically effective amount of the radioactive ligand to accelerate the clearance of any unbound bispecific affinity reagent from the subject's bloodstream and reduce the likelihood that the radioactive moiety will bind to bispecific affinity reagent that is not bound to CD45.

19. The method of claim 18, wherein the clearing agent is or comprises DOTAY, dextran-DOTAY, or NGB (biotinylated N-acetyl-galactosamine).

20. The method of claim 17, wherein the malignancy is a hematological malignancy.

21. The method of claim 20, wherein the hematological malignancy is defined as a leukemia, a myeloma, or a lymphoma.

22. The method of claim 21, wherein the leukemia is acute lymphocytic leukemia (ALL), myelodysplastic syndromes with excess blasts, acute myelogenous leukemia (AML), chronic lymphocytic leukemia (CLL), or chronic myelogenous leukemia (CIVIL).

23. The method of claim 20, wherein the malignancy is a T cell malignancy or a B cell malignancy.

24. The method of claim 17, wherein the radioactive ligand is or comprises yttrium-DOTA (Y-DOTA), or lutetium-DOTA (Lu-DOTA), or yttrium-DOTATE, or lutetium-DOTATE, or astatine-B10, or iodine-B10.

25. A nucleic acid comprising a sequence that encodes the bispecific affinity reagent of any one of claims 1-16.

26. A vector comprising the nucleic acid of claim 25.

27. The vector of claim 26, further comprising a promoter sequence operatively linked to the nucleic acid.

28. A cell comprising the nucleic acid of claim 25, or the vector of claim 26 or claim 27.

29. A method of producing the bispecific affinity reagent of any one of claims 1-16, comprising causing the expression of the nucleic acid of claim 25 in the cell of claim 28.
